(19) United States
(12) Patent Application Publication
(10) Pub. No.: US 2016/0296723 A1
(43) Pub. Date: Oct. 13, 2016

(54) MIXING CATHETER FOR TWO-PART SYSTEM

(71) Applicants: Rany Busold, Medford, MA (US); Jeffrey V. Groom, II, Medford, MA (US); Jennifer Mortensen, Athens, GA (US); Xuri R. Yan, Brighton, MA (US); Stephanie Webber, Brookline, MA (US)

(72) Inventors: Rany Busold, Medford, MA (US); Jeffrey V. Groom, II, Medford, MA (US); Jennifer Mortensen, Athens, GA (US); Xuri R. Yan, Brighton, MA (US); Stephanie Webber, Brookline, MA (US)

(21) Appl. No.: 15/093,264

(22) Filed: Apr. 7, 2016

Related U.S. Application Data
(60) Provisional application No. 62/144,058, filed on Apr. 7, 2015.

(51) Int. Cl. A61M 25/00; A61B 17/12

(52) U.S. Cl. CPC A61M 25/0074; A61M 25/0026; A61B 17/1219; A61B 17/12113

(57) ABSTRACT

Dual-lumen catheters for actively mixing two or more fluid components such that they react to form a more viscous pre-polymer formulation at or near the distal tip of the catheter are describe herein. The mixing dynamics within the dual-lumen catheter may be varied depending on the relative viscosity of each individual fluid component, as well as the viscosity of the resulting pre-polymer formulation.

### CROSS-REFERENCE TO RELATED APPLICATIONS

This application claims priority under 35 U.S.C. §119(e) to U.S. Provisional Application Ser. No. 62/144,058, filed on Apr. 7, 2015, herein incorporated by reference in its entirety. The entire disclosure of each of the foregoing applications is incorporated by reference herein for all purposes.

### FIELD OF THE INVENTION

The present disclosure relates to systems and methods for delivering polymer foams within a body lumen of a patient. More particularly, the present disclosure relates to a dual-lumen catheter for actively mixing two or more fluid components such that they react to form a more viscous pre-polymer formulation at or near the distal tip of the catheter.

### BACKGROUND

The expansive nature and highly customizable chemical and physical properties of polymer foams make them ideal for applications that require non-invasive delivery into spaces within the body. Polymer foams are typically delivered by mixing two (or more) fluid components within a catheter to form a more viscous pre-polymer formulation that flows into the patient and solidifies. However, the viscosities of the fluid components and resulting pre-polymer formulation are such that mixing within the catheter on the time and length scale required for efficient and accurate delivery to the patient remains a challenge.

Techniques for mixing fluids are generally separated into two categories: passive mixing and active mixing. Passive mixing relies entirely on the pressure of flowing fluids to create shear forces and increased interfacial areas to mix fluids together. This is unlike active mixing, which requires the input of additional energy to the system. While passive mixing systems tend to be less complex than their active mixing counterparts, the high delivery pressures required for passive mixing of fluids—especially viscous fluids—are often unsuitable for the delicate requirements of many in-situ applications. Active mixing systems are generally advantageous for such applications, in terms of both mixing efficiency and pressure requirements, because the energy provided to mix the fluids is supplied externally, thereby eliminating the need to increase the flow rate and/or pressure of either fluid stream to facilitate mixing.

Thus, there is a continued need for a mixing catheter that can actively combine two or more fluid components such that they react with minimal pressure to form a more viscous pre-polymer formulation at or near the distal tip of the catheter for delivery into a body lumen.

### SUMMARY OF THE INVENTION

The present disclosure describes systems and methods for actively mixing two or more fluid components such that they react to form a more viscous pre-polymer formulation at or near the distal tip of the catheter. The viscous pre-polymer formulation exits the catheter tip for delivery to a space inside the body, where it subsequently cures and/or crosslinks to form a polymer foam. The foam may be used for a variety of clinical applications including stabilizing organs, providing hemostasis and treating endoleaks prior to or following endovascular repair of abdominal aortic aneurysms.

In one aspect, the present disclosure relates to a mixing catheter, comprising a first lumen having a proximal end and a distal end; a second lumen having a proximal end and a distal end; a distal tip located distal to the first and second lumens, the distal tip including a mixing chamber in fluid connection with the first and second lumens; a mixing element disposed within the mixing chamber; and a power source configured to deliver mixing energy to the mixing element. The power source may be an external power source connected to the mixing element by, for example, a shaft (i.e., drive shaft), wire or fiber optic cable. The mixing energy may be mechanical energy, electrochemical energy acoustic energy, thermal energy (i.e., heat), radiofrequency radiation energy and/or ultraviolet light. The mixing element may include a variety of planar or helical designs with open or closed configurations, such as loop, hoops, paddles and the like, configured to rotate about an axis within (or just beyond) the mixing chamber. The mixing element may also include a wire configured to vibrate within the mixing chamber. The distal tip of the catheter may be expandable, for example, in response to the activation of the power source.

In another aspect, the present disclosure relates to a method of treating a patient, comprising: inserting, into a body of the patient, a distal end of a mixing catheter having first and second lumens and a distal tip located distal to the first and second lumens, the distal tip including a mixing chamber in fluid connection with the first and second lumens and a powered mixing element disposed within the mixing chamber; and flowing first and second fluid components through the first and second lumens, the powered mixing element and the distal tip of the catheter, thereby forming a viscous pre-polymer formulation within the body of the patient. The viscous pre-polymer formulation may react with a bodily fluid to form a polymer foam within the body of a patient. The distal tip of the catheter may be positioned within a cavity of body lumen of a patient, including for example, an aneurysm sac. The step of flowing the first and second fluid components may include activating a power source configured to rotate or vibrate the powered mixing element.

In another aspect, the distal segment of the catheter, which may vary in length, and is preferably 1.0 cm to 5.0 cm from the distal tip of the catheter to the portion exiting from the introducer sheath, may be made from an elastic material or structure similar to a stent, or pre-formed thin plastic that is initially in a collapsed form to allow placement of the catheter to the target location. For example, the catheter may have an outer diameter that allows it to fit through a 5-6 Fr introducer sheath. Once removed from the introducer sheath, the distal end of the catheter may then expand to reduce the pressures associated with delivery of the highly viscous pre-polymer formulation. In one aspect, pressurization of the catheter lumen from the flow of the pre-polymer formulation fully forces the distal segment to fully open such that the formulation flows into the body at a lower pressure. In another aspect, the distal segment may be made from a self-expanding elastic material or pre-formed thin plastic that fully opens when not constrained by a retaining sheath disposed on the outer surface of the catheter.

In another aspect, the drive shaft may be replaced by a magnetically driven mixing element. For example, the mixing element may include a magnetic portion capable of being driven by an electromagnetic coil positioned at or near the catheter tip.

In another aspect, the mixing element may be positioned outside the catheter (i.e., beyond the distal tip) to eliminate curing of the highly viscous pre-polymer formulation within the catheter. Positioning the mixing element outside of the catheter lumen may reduce the effects of unwanted curing within the catheter, thus keeping the catheter tip unobstructed and preventing potentially harmful pressure increases.

In yet another aspect, the present disclosure relates to a method of treating a patient, comprising the steps of: inserting a distal end of a mixing catheter into a body of a patient, wherein the mixing catheter includes first and second lumens and a distal tip located distal to the first a second lumens, the distal tip including a mixing chamber in fluid connection with the first and second lumens and a powered mixing element disposed within the mixing chamber; and flowing first and second fluid components through the first and second lumens, the powered mixing element and the distal tip of the catheter, thereby forming a viscous pre-polymer formulation within the body of the patient. The viscous pre-polymer formulation may react with a bodily fluid within the body of the patient. The distal tip of the catheter may be placed within a body cavity or lumen of the patient. The distal tip of the catheter may be positioned within an aneurysm sac. In one embodiment, the step of flowing the first and second fluid components includes activating a power source configured to rotate or vibrate the powered mixing element.

For the purposes of this disclosure, the terms “pre-polymer” and “pre-polymer formulation” may be used interchangeably to designate a polymer-based material resulting from the combination of two or more fluid components that is capable of further reaction in a vessel or cavity to form a polymer foam. Either, or both, of the two (or more) fluid components may include additives (e.g., catalysts, surfactants, solvents, diluents, cross-linkers, chain extenders, blowing agents, etc.) that react when mixed to form the “pre-polymer formulation.” Pre-polymer formulations may be designed to foam to a predetermined maximum volume based on the isocyanate content, hydrophilicity and catalyst. The foams formed from the pre-polymer may be bioresorbable or non-absorbable, and are biocompatible for the specific application. The polymer foams described herein may include, but are not limited to, any suitable foam formed in-situ from a one, two, or multi-part formulation as described in U.S. application Ser. No. 13/209,020, filed Aug. 12, 2011 and titled “In situ Forming Hemostatic Foam Implants,” U.S. application Ser. No. 12/862,362, filed Aug. 24, 2010 and titled “Systems and Methods Relating to Polymer Foams,” each of which are incorporated by reference herein for all purposes.

As used herein, a material is described as a “fluid” or “viscous fluid” if it is flowable, as is the case with, for example, fluid, semi-solid, and viscous materials. As used herein, a material is said to “foam” in that it undergoes a chemical and/or physical change that results in the formation of a solid, a semi-solid, or a more viscous fluid. A “fluid,” as that term is used in this disclosure, may comprise a singular polymer fluid, or may comprise a plurality of polymer fluid components.

As used herein, “viscosity” refers to the measure of a fluid's resistance to deformation by shear and/or tensile stress. As a fluid is forced through a tube, the pressure (i.e., force) required to overcome the friction between the fluid and the walls of the tube is proportional to the fluid's viscosity. In general, a fluid is referred to as “viscous” if its viscosity is substantially greater than the viscosity of water.

As used herein, the term “Reynolds number” refers to the ratio of inertial forces to viscous forces for a given fluid flow, and may be used to predict flow patterns in different fluid flow scenarios. In general, laminar flows characterized by smooth and constant fluid motion tend to occur at low Reynolds numbers where viscous forces are dominant, whereas turbulent flows characterized by chaotic eddies, vortices and related flow instabilities tend to occur at high Reynolds numbers where inertial forces are dominant.

As used herein, “active mixing” refers to the application of external energy to fluid components to drive mixing. In one embodiment, the external energy may be applied in the form of electromechanical energy or ultrasonic energy. External energy may be provided by, for example, a small electric motor connected to a drive shaft that extends the length of the catheter. In another embodiment, the external energy may be provided by a user in the form of a hand-powered device using mechanical leverage. The distal end of the drive shaft may include a tip (i.e., hoop, impeller etc.) that creates shear forces between the two (or more) fluid components when rotated. The tip may be positioned anywhere within a mixing chamber positioned between distal ends of the first and second lumens and the distal tip of the catheter. In another embodiment, the external energy may be applied in the form of pneumatic energy such as, for example, compressed/pressurized air or inert gas.

As used herein, the terms “injected,” “deposited,” “delivered” and the like, are used to indicate that the pre-polymer formulation is placed via a delivery catheter at a target location within a patient's body.

### DRAWINGS

Non-limiting embodiments of the present disclosure will be described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure.

FIG. 1 is a schematic view of a T-mixer tip catheter in which passive mixing occurs as fluid streams from two lumens of a dual-lumen catheter collide in a mixing chamber perpendicular to flow.

FIG. 2 is a schematic view of an active mixing dual-lumen catheter in which separate fluid components are combined by a mixing element disposed within a mixing chamber positioned at a distal end of the catheter, in accordance with an embodiment of the present disclosure.

FIGS. 3A-F depict various designs for mixing elements, in accordance with embodiments of the present disclosure.

FIG. 3G depicts a single “hoop” mixing element configured to rotate about its axis within a catheter lumen, in accordance with an embodiment of the present disclosure.

FIG. 4 is a schematic view of an ultrasonic active mixing dual-lumen catheter, in accordance with an embodiment of the present disclosure.

FIGS. 5A-B are schematic views of an elastic-tipped dual-lumen catheter in unexpanded (5A) and expanded (5B) configurations, in accordance with an embodiment of the present disclosure.

FIGS. 6A-B are schematic views of a flared-tipped dual-lumen catheter in unexpanded (6A) and expanded (6B) configurations, in accordance with an embodiment of the present disclosure.

### DETAILED DESCRIPTION OF THE PREFERRED EMBODIMENTS

The present disclosure relates generally to systems and methods for delivering polymer foams within a body lumen of a patient. More particularly, the present disclosure relates to a dual-lumen catheter configured to actively mix two or more separate fluid components, at or near that distal tip of the catheter, such that they react to form a pre-polymer formulation that is more viscous than either individual fluid component. The viscosity of each individual fluid component may range from low to moderate viscosity. Accordingly, the two (or more) fluid components may have similar or disparate viscosities. The mixing dynamics within the dual-lumen catheter may be varied depending on the relative viscosity of each individual fluid component, as well as the viscosity of the resulting pre-polymer formulation. As will be understood by those of skill in the art, the pre-polymer formulation may react to form a polymer foam upon exiting the catheter tip. Alternatively, the pre-polymer formulation may already be in the form of a polymer foam that cures and/or crosslinks to change its final configuration upon exiting the catheter tip.

Generally, in-situ foaming formulations for use with the catheters of the present disclosure are provided as one-part or two-part formulations that react to form a polyurethane foam. A one-part pre-polymer formulation typically consists of an isocyanate-functionalized pre-polymer that may optionally contain multiple polymer species, catalysts, surfactants, chain extenders, cross-linkers, pore openers, fillers, plasticizers and/or diluents. The pre-polymer forms a foam upon interacting with an aqueous environment (e.g., blood, water, and/or saline) during, or immediately following, delivery into the patient. Preferably the blood, water, or saline controls the volume of expansion such that foaming stops once the aqueous solution is depleted from the body lumen in which the formulation is introduced. A two-part pre-polymer formulation is generally formed by combining two (or more) fluid components which are stored separately, then mixed and/or aerated and delivered to a site in the body where they react to form a foam. The fluid components typically include separate functionalized molecules which react to form a cross-linked polymer, for instance a polyol-functionalized pre-polymer and an isocyanate cross-linker, and optionally include additives which modify the physical or chemical features of the foam which is generated by the combination of the two components.

In one embodiment, the present disclosure provides a dual-lumen catheter that actively mixes two (or more) fluid components at or near the distal end of the catheter to form a more viscous pre-polymer formulation that is injected or deposited at a desired location within the body of a patient. As used herein, a “dual-lumen catheter” refers to a device that can be introduced into a patient's body and positioned adjacent to a target location, and comprises at least two lumens of an appropriate size, shape or configuration (e.g. coaxial, or side-by-side) for the movement of separate fluid components into a common mixing chamber where the fluids react to form a more viscous pre-polymer formulation. The pre-polymer formulation then flows from the mixing chamber into the patient where it cures and/or crosslinks to form a polymer foam. Depending on the viscosities of the individual fluid components and resulting pre-polymer formulation, a hand-powered syringe-assist, pneumatic pressure pump, or other device may be used to control the flow rate of each fluid component into the mixing chamber, and the delivery of the pre-polymer formulation into the patient. Any means well-known in the art may be used to deploy the dual-lumen catheter to the target site, including but not limited to, guide wires, guide catheters, introducers, endoscopes or percutaneous needles. The embodiments of the disclosure may also include any additional equipment necessary to deliver the foam to the target site, including but not limited to, additional catheters, guide wires, needles, positioning equipment, foam component containers, dispensing and metering systems and introducer sheaths.

### Active Mixing at the Catheter Tip

Two primary approaches are available to address the problems associated with mixing fluid components with similar or disparate viscosities within a dual-lumen catheter. The first approach is to viscify (i.e., mix, blend, combine etc.) the fluid components as close to the catheter tip as possible, thereby allowing the more viscous pre-polymer formulation to exit the catheter tip as it is being formed. Allowing the more viscous pre-polymer formulation to exit the catheter tip as the fluid components are being mixed not only reduces the pressure within the catheter, but also minimizes the potential for polymerized foam components to accumulate within and occlude the catheter lumen. In one embodiment, the fluid components are viscified less than 1.0 cm from the catheter tip. In one embodiment, sufficient mixing of fluid components on the time and length scale required for forming a more viscous pre-polymer formulation is achieved by a mixing element located within the distal tip of the catheter.

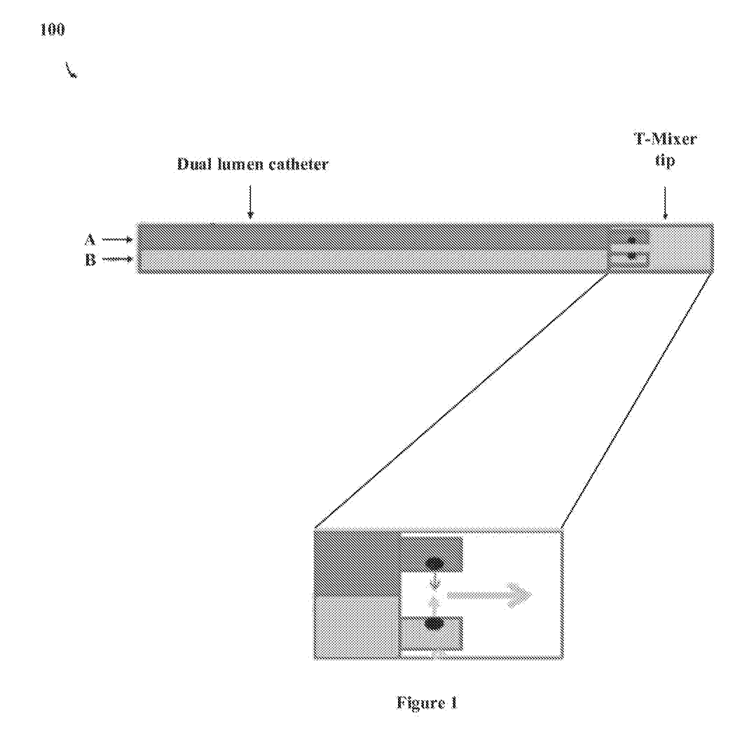

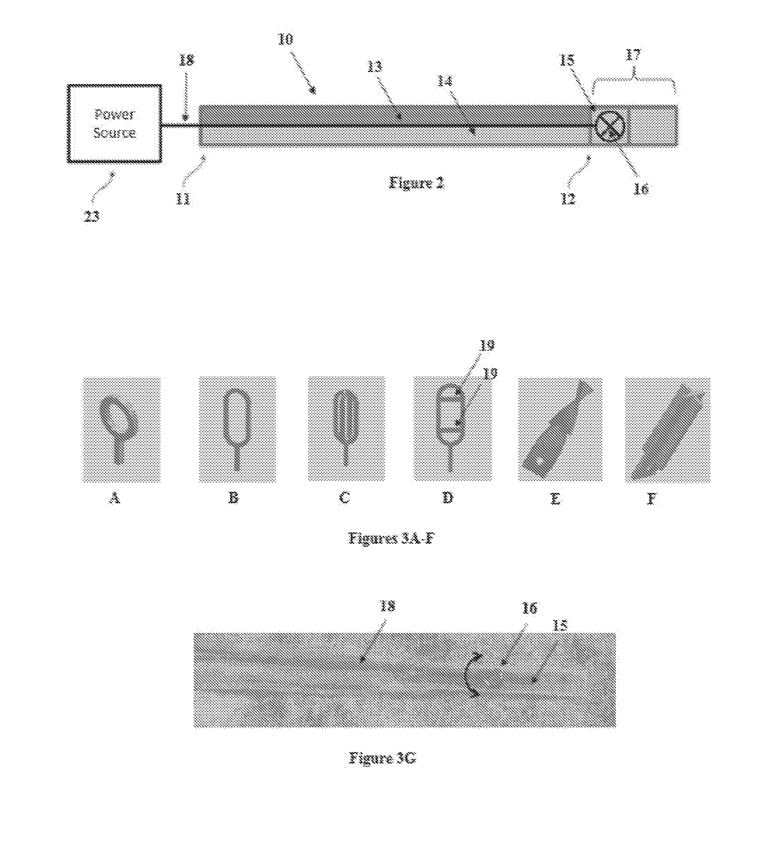

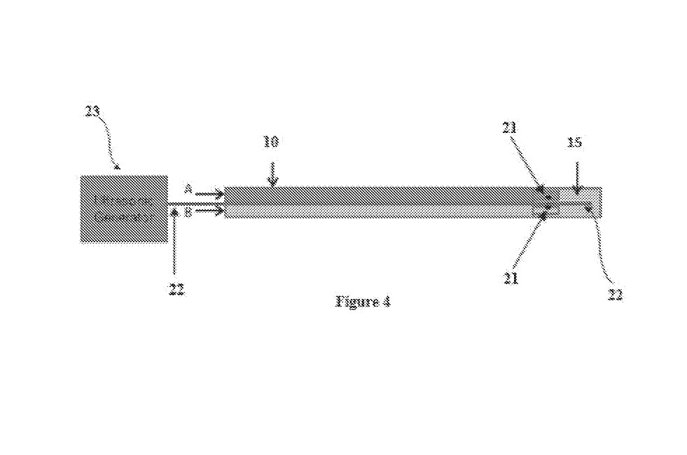

Passive mixers such as static helical mixers, lamination mixers and T-mixers have been shown in microfluidics research to provide mixing at low Reynolds numbers on a small length and time scale. FIG. 1 depicts a passive T-mixer tip catheter in which each of the two lumens of the dual-lumen catheter terminate in a segment that forces fluids flowing therethrough to be expelled through opposed openings perpendicular to their direction of flow. The fluid streams exiting from each opposed opening are aligned such that they collide within the mixing chamber to undergo rapid mixing. By way of comparison, FIG. 2 depicts one embodiment of an active mixing dual-lumen catheter 10 that includes proximal 11 and distal 12 ends with first and second lumens 13, 14 extending therethrough. Distal tip 17 of catheter 10 includes a mixing chamber 15 fluidly connected to the distal end 12 of first and second lumens 13, 14 to receive fluid components flowing therethrough. A mixing element 16 is disposed within mixing chamber 15 to actively viscify the fluid components such that they react to produce the more viscous pre-polymer formulation. In one embodiment, mixing element 16 is connected to an external power supply 23 by shaft 18 that extends the length of the dual-lumen catheter. As best depicted in FIG. 3E, shaft 18 and mixing element 16 may be formed from a continuous piece of material that is bent into a loop at its distal end. In addition to being formed as an integral part of shaft 18, mixing element 16 may be formed separately and attached to a distal end of shaft 18.

In one embodiment, power supply 23 provides mechanical power that actuates shaft 18 to rotate about its axis. Shaft 18 may be formed from a variety of suitable materials with a sufficient torque ratio (e.g., 1:1 torque ratio) to prevent binding. Non-limiting examples of such materials include a single piece of 316 stainless steel or a braided torque wire. In one embodiment, shaft 18 extends the length of the dual-lumen catheter between the first and second lumens through a third lumen (not shown). In a preferred embodiment, shaft 18 and mixing element 16 may be removed from the third lumen and replaced with a guide wire for delivery of the dual-lumen catheter within the patient. Once the dual-lumen catheter is properly positioned the guide wire may be withdrawn from the third lumen and replaced with shaft 18 and attached mixing element 16. In one embodiment, shaft 18 extends the length of the catheter through one of the first or second lumens in a dual-lumen catheter. The dual-lumen catheter and mixing element 16 may be designed to allow removal and insertion of the mixing element 16 through this lumen, allowing for guide wire delivery of the catheter within the patient.

FIG. 3A depicts one embodiment of a mixing element comprising a single “hoop” (i.e., circle, loop, ring etc.) that rotates about its axis within mixing chamber 15 to create shear forces that viscify the two (or more) fluid components therein. The outer diameter (OD) of mixing element 16 is preferably dimensioned to match the inner diameter (ID) of mixing chamber 15 in order to prevent stagnant flows against the inner wall of the mixing chamber. As best illustrated in FIG. 3G (see arrows), mixing element 15 is configured to rotate about its axis in a variety of patterns, including a series of alternating back-and-forth movements (e.g., 90 degrees, 180 degrees etc.) or as a series of complete revolutions (e.g., 360 degrees). Examples of open design mixing elements include those used for mixing cake batter (e.g., anchor or helical ribbon designs) or similar low Reynolds number batch mixing designs as will be known in the art. By way of example, FIG. 3B depicts one embodiment of a mixing element in which the “hoop” design of FIG. 3A is elongated to provide an oblong (i.e., oval) shape that enhances mixing by increasing the surface area through which the fluid components pass as the mixing element rotates. Mixing efficiency may be further enhanced by incorporating structures into the mixing element that increase the shear forces within the mixing chamber. For example, FIG. 3C depicts an embodiment in which the planar design of FIG. 3B is modified to include a second oblong loop perpendicular to the plane of the first. Similarly, FIG. 3D depicts an embodiment in which the oblong loop of FIG. 3B is modified to include horizontal cross-pieces 19. In another embodiment, the mixing element may be made in a closed configuration that increases the interfacial area of the fluids components by effectively splitting and recombining the fluids as they pass through the rotating mixing element. For example, FIG. 3E depicts an embodiment in which the mixing element includes a 2-turn helical design, while FIG. 3F depicts a mixing element that includes four “paddles” that intersect at approximately 90° angles. Without wishing to be bound by any theory, the number of “turns” or “paddles” in these designs and the speed of rotation will impact the efficiency of mixing.

In another embodiment, acoustic stirring may be used to mix the fluid components by introducing ultrasonic wave pulses (i.e., vibrations) into the mixing chamber to create shear forces within the mixing chamber. As depicted in FIG. 4, power supply 23 may include an ultrasonic frequency generator connected to an ultrasonic mixing wire 22 that travels the length of the dual-lumen catheter and extends into mixing chamber 15 of a passive T-mixer catheter tip, as shown in FIG. 1. The dual-lumens of the ultrasonic mixer include end segments having openings 21 that force the fluid components out perpendicular to flow where they collide in the presence of ultrasonic mixing energy emitted from ultrasonic mixing wire 22. It should be appreciated that ultrasonic mixing is not limited to T-mixer catheter tip designs, but may be used with any of the dual-lumen catheters described herein. For example, the dual-lumens may force the fluids out parallel to each other, with the ultrasonic mixing wire 22 located at the interface of the fluids with in the mixing chamber. Alternatively, the ultrasonic mixing wire 22 may be extend beyond (i.e., distal to) the mixing chamber at the distal tip of the catheter such that ultrasonic energy is applied to the pre-polymer formulation as it exits the catheter tip.

In one embodiment, power supply 23 (e.g., battery) causes a piezoelectric membrane on the tip of the ultrasonic probe 22 to vibrate at ultrasonic frequencies. Cavitation induced by these ultrasonic vibrations results in very high localized shearing within the mixing chamber to viscify the fluids. In another embodiment, ultrasonic vibrations may be introduced into the fluid components prior to their introduction into the dual-lumen catheter. Such ultrasonic vibrations would propagate a pressure wave through the fluid components to facilitate their transport into the distal mixing chamber. Upon exit into the mixing chamber, the pressure waves would provide shearing forces at the interface of the two fluids to promote mixing.

Depending on the specific mixing requirements, mixing within the catheter tip may be further optimized by individually adjusting the fluid exit velocities and/or pressure of each fluid component. For example, the inner diameter of opposed openings 21 may be increased or decreased to generate appropriate fluid exit velocities. In one embodiment, the openings may have a diameter from about 0.05 mm to about 0.5 mm, and preferably from about 0.05 mm to about 0.25 mm. Similarly, fluid pressures may be adjusted by varying the thickness of the wall through which opposed openings 21 extend, thereby increasing the distance that each fluid travels perpendicular to flow. Additionally, the distance (i.e., length) from the point of mixing to the catheter exit, and the distance between the opposed openings 21, may be adjusted to further regulate the amount of mixing that occurs.

### Expandable Catheter Tips

A second approach to address the problems associated with mixing fluid components within a catheter is to increase the diameter of the catheter tip to counteract pressure increases resulting from increased fluid viscosity. Because pressure is proportional to the inverse of the radius (r̂4), even a slight increase in the diameter of the catheter tip provides a substantial reduction in the overall pressure exerted on the system. However, in order to facilitate introduction within a patient in as minimally invasive of a manner as possible, the dual-lumen catheter of the present disclosure preferably includes a smooth outer surface with constant outer diameter. This requires that any increase in the diameter of the catheter must occur after the catheter tip has been positioned within the target body lumen of the patient.

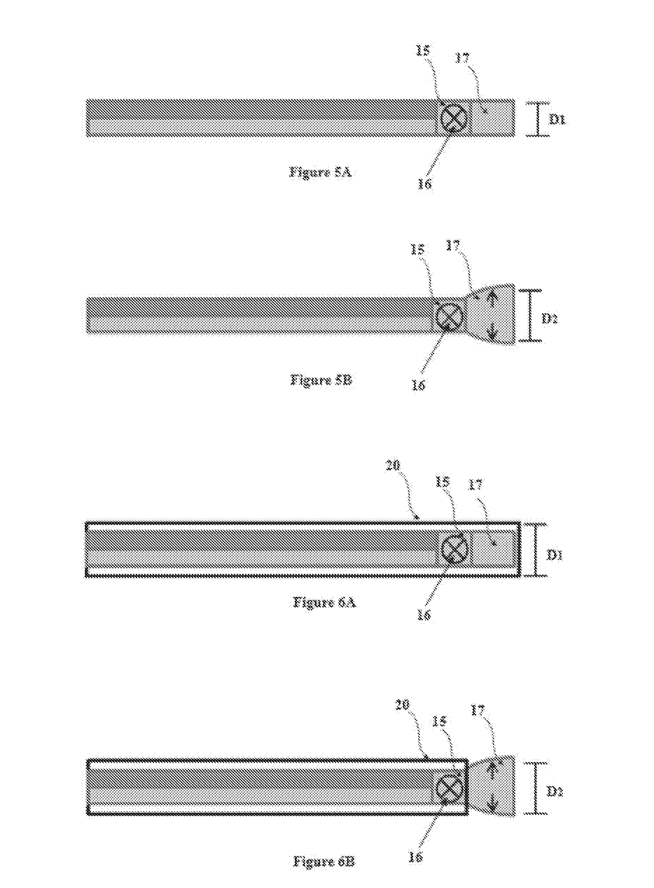

FIGS. 5A-B depict a dual-lumen catheter comprising a distal tip 17 made of an elastic material capable of expanding in response to outward pressure. At rest, distal tip 17 has an outer diameter D₁ that matches the outer diameter of the dual-lumen catheter. As the fluid components are combined within mixing chamber 15, the resulting pre-polymer formulation dramatically increases the pressure within distal tip 17. The force (FIG. 5B; see arrows) generated by the pre-polymer formulation causes the elastic material of distal tip 17 to expand to outer diameter D₂. When mixing of the fluid components within mixing chamber 15 is complete, the elastic material of distal tip 17 returns to resting diameter D₁, allowing the dual-lumen catheter to be withdrawn from the patient. In one embodiment, the elasticity of the material may be tailored to allow a specific and robust amount of stretching to occur based on the pressure exerted. In one embodiment, the distal tip may be 0.1 cm to 10.0 cm in length, but more preferably less than 5.0 cm in length, even more preferably less than 3.0 cm in length and even more preferably less than 2.0 cm in length.

FIGS. 6A-B depict a dual-lumen catheter comprising a distal tip 17 made of a self-expanding elastic material housed in sheath 20. Once the dual-lumen catheter is positioned at the appropriate location within a patient, sheath 20 is retracted in a proximal direction relative to the dual-lumen catheter. When released from sheath 20, the self-expanding material of distal tip 17 transitions from a restrained configuration with outer diameter D₁, to a flared configuration with an outer diameter D₂. When mixing of the fluid components within mixing chamber 15 is complete, distal tip 17 is returned to the restrained configuration by advancing sheath 20 in the distal direction. The dual-lumen catheter may then be withdrawn from the patient. In one embodiment, the distal tip may be 0.1 cm to 10.0 cm in length, but more preferably less than 5.0 cm in length, even more preferably less than 3.0 cm in length and even more preferably less than 2.0 cm in length.

A variety of elastic materials (expandable and/or self-expandable) such as polyurethanes, thermoplastics, elastomers, fiber-reinforced elastomers, latex/plastic polymer blends, silicone, vinyl, foams and rubbers may be used to form the expandable tip.

### Heating at the Distal Tip

In another embodiment, one or more fluid components may be converted to a more viscous pre-polymer formulation by the application of heat at the catheter tip. Heat may be applied 0.5-10.0 cm from the tip, and is preferably applied less than 3.0 cm proximal to the distal end of the catheter tip. Further cross-linking of the formulation may then occur in the body cavity to solidify the material and prevent continued flow. In one embodiment, a heat source is connected to an insulated wire that runs the length of the catheter. The distal end of the wire is uninsulated such that heat is transferred to the formulation to initiate curing. The wire may be formed into any shape or pattern necessary to provide adequate heat transfer. For example, the wire may be coiled around the distal tip, either internal or external or embedded within the wall of the lumen. In one embodiment, radiofrequency (RF) radiation is used as the heat source. An RF energy source transmits energy of a desired frequency along the wire to emit radiation of a desire frequency at the distal 3.0 cm of the catheter to initiate curing of the formulation. In another embodiment, ultraviolet (UV) or visible light is used as the energy source to initiate curing of the formulation at the catheter tip. For example, a power supply may be connected to a light emitting diode (LED) attached to a thin fiber optic cable that runs the length of the catheter and terminates at the distal end of the catheter tip. Light of a specific wavelength provided by the LED would then exit the cable and cure the formulation. The wavelength and intensity of the light as well as the numerical aperture of the cable may be selected to provide an ideal curing profile of the formulation.

While several embodiments of the present disclosure have been described and illustrated herein, those of ordinary skill in the art will readily envision a variety of other means and/or structures for performing the functions and/or obtaining the results and/or one or more of the advantages described herein. Each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that the disclosure may be practiced otherwise than as specifically described. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A mixing catheter, comprising:
a first lumen having a proximal end and a distal end;
a second lumen having a proximal end and a distal end;
a distal tip located distal to the first and second lumens, the distal tip including a mixing chamber in fluid connection with the first and second lumens;
a mixing element disposed within the mixing chamber; and
a power source configured to deliver mixing energy to the mixing element.

2. The mixing catheter of claim 1, further comprising a passive mixing structure.

3. The mixing catheter of claim 1, wherein the mixing energy is mechanical or acoustic energy.

4. The mixing catheter of claim 1, wherein the mixing energy is electromechanical or radiofrequency energy.

5. The mixing catheter of claim 1, wherein the mixing energy is heat or ultraviolet light.

6. The mixing catheter of claim 1, wherein the mixing element is connected to the power source by at least one of a shaft, a wire or a fiber optic cable.

7. The mixing catheter of claim 1, wherein the mixing element is configured to rotate about an axis within the mixing chamber.

8. The mixing catheter of claim 1, wherein the mixing element is configured to vibrate within the mixing chamber.

9. The mixing catheter of claim 1, wherein the distal tip is expandable.

10. The mixing catheter of claim 9, wherein the distal tip expands in response to activation of the power source.

11. A method of treating a patient, comprising the steps of:
inserting, into a body of the patient, a distal end of a mixing catheter having first and second lumens and a distal tip located distal to the first and second lumens, the distal tip including a mixing chamber in fluid connection with the first and second lumens and a powered mixing element disposed within the mixing chamber; and
flowing first and second fluid components through the first and second lumens, the powered mixing element and the distal tip of the catheter, thereby forming a viscous pre-polymer formulation within the body of the patient.

12. The method of claim 11, wherein the viscous pre-polymer formulation reacts with a bodily fluid within the body of a patient.

13. The method of claim 11, wherein the distal tip of the catheter is positioned within an aneurysm sac.

14. The method of claim 11, wherein the distal tip of the catheter is placed within a body cavity or lumen of the patient.

15. The method of claim 11, wherein the step of flowing the first and second fluid components includes activating a power source configured to rotate or vibrate the powered mixing element.

16. The method of claim 11, further comprising the step of applying heat or light energy to the viscous pre-polymer solution within the body of the patient, thereby curing the viscous pre-polymer solution.

17. A system for treating a patient, comprising:
first and second fluid vessels containing fluid components, respectively, the first and second fluid components configured to form a viscous pre-polymer formulation within a body of the patient when mixed;
a catheter having proximal and distal ends and comprising:
first and second lumens fluidly connected to the first and second fluid vessels;
a distal tip located distally of the first and second lumens, the distal tip including a mixing chamber fluidly connected to the first and second lumens; and
a mixing element disposed on or in the distal tip,
wherein the viscous pre-polymer formulation is configured to react within the body of the patient.

18. The system of claim 17, wherein the mixing element is at least one of a T-mixer, a helical impeller, a heating element, an electrode and a fiber-optic light source.

19. The system of claim 17, wherein the distal tip is expandable.

20. The system of claim 17, further comprising one of a fiber optic light source and a heating element configured to apply energy to an exterior of the catheter, thereby curing the viscous pre-polymer solution.
